(19) United States
(12) Patent Application Publication
(10) Pub. No.: US 2017/0074316 A1
(43) Pub. Date: Mar. 16, 2017

(54) JOINT APPARATUS, AND TRAINING DEVICE, RING TYPE JOINT STRUCTURE, CONSTRUCTION TOY, AND ARTIFICIAL JOINT USING SAME

(71) Applicants: Sug-Whan KIM, Gyeonggi-do (KR); SphereDyne Co., Ltd., Seoul (KR)

(72) Inventor: Sug-Whan KIM, Gyeonggi-do (KR)

(21) Appl. No.: 15/123,462

(22) PCT Filed: Feb. 26, 2015
(86) PCT No.: PCT/KR2015/001876

§ 371 Date: Sep. 2, 2016

(30) Foreign Application Priority Data
Mar. 3, 2014 (KR) 10-2014-0024836

(51) Int. Cl. F16C 11/06; A61F 5/01; B25J 17/02

(52) U.S. Cl. CPC F16C 11/06; B25J 17/0275; A61F 5/0125

(57) ABSTRACT

The present invention relates to an elastic joint apparatus (a flexure mechanism) that can be used for various purposes, such as an industrial purpose, a medical purpose, a training purpose, etc regarding human bodies, animals, mechanical devices, and products. More particularly, the present invention relates to an elastic joint apparatus (a flexure mechanism) in which a joint connection means inserted and mounted into a joint means enables linear movements in X-axis, Y-axis and Z-axis directions and rotational movements in altitude, azimuth and axial rotation directions by means of the bending and elastic force of an elastic means, and in which the joint connection means is configured to be separated from and coupled to the joint means by the elastic force of the elastic means in proportion to applied force.

### FIELD OF THE DISCLOSURE

The present invention relates to a joint apparatus that can implement the functions of a joint of a human body, an animal or a machine and, thus, can be used for various purposes, such as an industrial purpose, a medical purpose, a training purpose, etc. More particularly, the present invention relates to an elastic joint apparatus in which a joint connection means inserted and mounted into a joint means enables linear movements in X-axis, Y-axis and Z-axis directions and enables rotational movements around an X axis, an Y axis and an Z axis, i.e., rotational movements in altitude, azimuth and axial rotation directions, by means of the bending and elastic force of an elastic means, and in which the joint connection means is configured to be separated from and coupled to the joint means by applied force. The joint apparatus can simulate various joint functions such as the functions of being bent, being restored, and being extended, and thus can be used in an animal, a human body, a mechanical apparatus, and an industrial product that require a joint function.

### BACKGROUND OF THE DISCLOSURE

In general, the functions of a joint include: 1) the function of transferring force from one side to the other side; 2) the function of changing the direction in which force is transferred when necessary; 3) the function of performing movement in space between one side and the other side, i.e., the function of performing predetermined direction movements of 3-direction linear movements and 3-direction rotational movements and restoration of a location; 4) the function of enabling one side and the other side to form a single structure in the state of being combined; 5) the function of remaining a single structure when external force is not applied and being separated when external force whose strength is equal to or higher than specific strength is applied; and 6) the function of being recombined to perform original functions after the joint has been separated. These joint functions may be applied to an animal, a human body, a mechanical device, or various types of industrial products in a wide range. Examples of the application include the following:

martial arts training equipment for punching, kicking, bending, twisting, pulling, pushing, etc.;

artificial joints for a human body and an animal, such as a hip joint, a knee, a shoulder, an arm, etc.;

various types of health equipment for muscle strengthening training;

industrial furniture, such as chair legs, regions connecting a seat and legs, bed legs, mattress springs, etc.;

rehabilitation aid tools, external structure strengthening exoskeletons, and weak muscle strength reinforcing skeleton structures;

robot joints, crash dummies, and various types of mechanical joints;

load measuring devices, such as multi-axis load cells;

construction toys, plastic models, etc. such as Lego models;

pipe coupling structures;

infrastructure interfaces of buildings or mechanical equipment support structures, vibration isolation structures, bendable streetlights, utility poles, etc.;

The martial arts training equipment corresponding to one of the above-described application fields has a disadvantage in that training apparatuses for martial arts or sports (fighting sports, such as wrestling, Judo, etc.) are not suitable for training for bending, choking, and twisting, other than training for hitting, because they have patterned protrusions extending from fixed locations, like Muk Yan Jong, and thus the reactions of target objects to the movements of a human body are not accurately transferred during training. In order to overcome this disadvantage, recently, an installation target object that is composed of a plurality of segment bodies configured to be attached to and detected from the joint regions of a human body in order to more realistically simulate the height, shape and function of the human body has been used.

However, this installation target object configured to simulate the shape of a human body and composed of the plurality of segment bodies is configured such that the plurality of segment bodies are simply combined to be rotatable with respect to each other, and thus has disadvantages in that the installation target object cannot simultaneously or sequentially simulate the various movements, such as bending, choking, twisting, pressing, pulling, etc., of a human body and in that an effect that is applied to a counterpart by hitting force cannot be realistically sensed. That is, this installation target object has a disadvantage in that during use, it is difficult to have a realistic sense of hitting or the degrees of reaction to various actions, such as bending and choking, or the degrees of reaction to the forms in which force is transferred are unrealistic, and, thus, the effect of training felt by a trainee falls short of his or her expectation. In particular, existing martial arts training apparatuses are not equipped with devices capable of implementing all the above-described six joint functions.

Meanwhile, conventional medical artificial joints are used with a focus on the facts that principal mechanical components are screwed into holes formed in a bone and a joint connection part is configured not to be separated as much as possible. Accordingly, once an artificial joint has been mounted, it is burdensome and technically difficult to remove the joint. That is, the artificial joint can be removed, replaced or maintained/repaired only by a surgical operation that exerts an influence on a bone of a patient.

Meanwhile, conventional construction toys maintain their shapes in such a way that a plurality of pieces is assembled through fitting coupling between a male joint portion and a female joint portion. Although the conventional construction toys can freely perform one degree of freedom angular movement, the conventional construction toys are problematic in that two degrees of freedom angular movement is limited and three or more degrees of freedom linear and rotational movement cannot be performed.

### PRIOR ART

(Patent document 1) Korean Patent No. 10-1132806

(Patent document 2) Korean Patent Application Publication No. 10-20-120020727

### SUMMARY

### Technical Problem

The present invention is proposed to overcome the above-described problems, and an object of the present invention is to provide a joint apparatus, which enables 6-degree-of-freedom movement, which can be separated when appropriate external force is applied, and which can be easily mounted without using a special tool.

Furthermore, an object of the present invention is to provide an elastic joint apparatus, which reacts in proportion to the magnitude of external force, and which is restored when external force is removed.

Moreover, an object of the present invention is to provide a joint apparatus, which can be applied to various fields, such as an industrial robot field, a medical joint field for rehabilitation, etc.

### Technical Solution

In order to accomplish the above objects, the present invention provides a joint apparatus, including: a joint means; an elastic means formed on one side of the joint means; and a joint connection means inserted and mounted into the joint means and configured to directly come into contact with and be held by the elastic means.

Preferably, a reception member configured to accommodate the joint connection means is disposed in the joint means.

Preferably, a guide member configured to guide the reception member through movement is fastened to and mounted in the joint means.

Preferably, the guide member and the reception member are connected by an elastic element.

Preferably, an elastic means opening is formed in the elastic means, and the joint connection means is inserted into the joint means via the elastic means opening.

Preferably, the joint connection means inserted into the joint means is supported through equilibrium of anti-elastic forces of the elastic means and the elastic element that act in opposite directions.

Preferably, the contact surfaces of the joint connection means and the reception member have corresponding male and female shapes or corresponding engaged shapes.

Preferably, the elastic means includes a plurality of elastic means units, and elastic means cutout slits are formed between the adjacent elastic means units.

Preferably, the elastic means has one or more bent portions so that the joint connection means can be easily inserted into the joint means.

Preferably, the elastic means enables elastic deformation in 3 degrees of freedom linear movement directions and 3 degrees of freedom rotational movement directions, and the joint connection means enables 3 degrees of freedom linear movements and 3 degrees of freedom rotational movements with respect to the joint means, thereby enabling 6 degrees of freedom movements.

A composite joint structure having a linear, surface, or three-dimensional structure can be formed through the coupling of connection links or connection rods.

Furthermore, the present invention provides a ring-shaped joint structure, including: two holder rings configured to have different diameters; a disk configured to have a diameter lower than those of the holder rings; and a protrusion configured to protrude perpendicularly from the disk; wherein by the joint apparatus the two holder rings are coupled and the holder ring having a smaller diameter is coupled to the disk, and the elastic means of the joint apparatus enables elastic deformation in 3 degrees of freedom linear movement directions and 3 degrees of freedom rotational movement directions.

Furthermore, the present invention provides training equipment, various types of dolls, and industrial machines and products to which one or more joint apparatuses according to the present invention are applied.

One or more sensor(s) may be mounted on the joint means or the elastic means, and then may detect applied force or strain.

Furthermore, the present invention provides a construction toy to which the joint apparatus according to the present invention is applied.

Furthermore, the present invention provides an artificial joint to which the joint apparatus according to the present invention is applied.

Furthermore, the present invention provides an artificial joint, including: a first target object; a skeleton-mounted ring configured to surround and fasten the first target object; a second target object; a skeleton-mounted disk configured to fasten the second target object; a joint connection means located between the first target object and the second target object; a reception member configured to accommodate the joint connection means between the first target object and the second target object; first elastic members radially coupled along the internal circumferential surface of the skeleton-mounted ring, and second elastic members radially coupled along the outer circumferential surface of the skeleton-mounted ring; and third elastic members radially coupled along the internal circumferential surface of the skeleton-mounted disk, and fourth elastic members radially coupled along the outer circumferential surface of the skeleton-mounted disk; wherein the first elastic members and the fourth elastic members are coupled to the joint connection means, and the second elastic members and the third elastic members are coupled to the reception member.

Preferably, the joint connection means includes a joint connection means head and a joint connection rod; and the reception member includes a receptor and a receptor support.

Preferably, the first elastic members, the second elastic members, the third elastic members, and the fourth elastic members are each plural in number.

Preferably, the first elastic members hold the joint connection rod; the second elastic members hold the receptor; the third elastic members hold the receptor support; and the fourth elastic members hold the joint connection means head.

### Advantageous Effects

The joint apparatus according to the present invention has the advantage of enabling linear and rotational movements in directions in space because the joint connection means inserted and mounted into the joint means enables linear movements in X-axis, Y-axis and Z-axis directions and rotational movements around an X axis, an Y axis and an Z axis in proportion to force externally applied in the state of being supported by the elastic force of the elastic means and, thus, 6 degrees of freedom for movement can be achieved.

Furthermore, the joint apparatus according to the present invention has the advantage of being applied to various types of sports equipment and apparatuses and thus maximizing martial arts and exercise training effects.

Furthermore, the joint apparatus according to the present invention has the advantage of being favorable for repetitive use because the joint connection means separated from the joint means can be easily inserted and mounted into the joint means.

Furthermore, the joint apparatus according to the present invention has the advantage of being usable for an industrial robot field, a medical joint mechanism field for rehabilitation, etc. because the joint apparatus is simple and can simulate the joint functions of a human body.

Moreover, the joint apparatus according to the present invention has the advantage of being usable for all types of industry products that require a flexible support structure.

### BRIEF DESCRIPTION OF THE DRAWINGS

FIG. 1 is a sectional view of a joint apparatus according to a first embodiment of the present invention;

FIG. 2 is a perspective view showing the body of the joint apparatus of the first embodiment shown in FIG. 1;

FIGS. 3 and 4 are a perspective view and a front view showing the cutout slits of the joint apparatus according to the first embodiment shown in FIG. 1;

FIG. 5 is a sectional view showing the guide member of the joint apparatus according to the first embodiment of the present invention;

FIGS. 6 and 7 are views showing embodiments of the joint connection means and reception member of the joint apparatus according to the first embodiment of the present invention;

FIGS. 8 and 9 are views showing various shapes of male and female heads;

FIG. 10 is a view showing various shapes of a connection rod;

FIGS. 11 and 12 are views showing various shapes of an elastic means;

FIG. 13 is a view showing applications of elastic means having various shapes;

FIG. 14 is a view showing various forms according to a second embodiment of the present invention;

FIG. 15 is a view showing various forms according to a third embodiment of the present invention;

FIGS. 16 and 17 are views showing examples in each of which a plurality of joint connection means heads is formed according to a fourth embodiment of the present invention;

FIGS. 18 and 19 are views showing joint apparatuses according to a fifth embodiment of the present invention;

FIGS. 20 to 23 are views showing examples in which joint apparatuses according to the present invention are connected in various forms;

FIG. 24 is an exploded perspective view of a sixth embodiment in which joint apparatuses according to the present invention are applied to one or more joint regions of a human body or various types of dummies and dolls;

FIGS. 25 to 27 are views showing examples of use in which the joint apparatuses according to the present invention are applied to training equipment;

FIG. 28 is a view showing an example of use in which a load cell is mounted on the joint apparatus according to the present invention;

FIG. 29 is a view showing an example of use in which the joint apparatus according to the present invention is applied to a construction toy;

FIG. 30 is a view showing examples of use in which the joint apparatus according to the present invention is applied to an exoskeleton and a muscular strength reinforcement or strengthening structure; and

FIG. 31 is a view showing examples of use in which the joint apparatus according to the present invention is applied to a cut or damaged internal joint of a human body.

### DETAILED DESCRIPTION

A joint apparatus according to the present invention is described in detail with reference to the accompanying drawings for each embodiment.

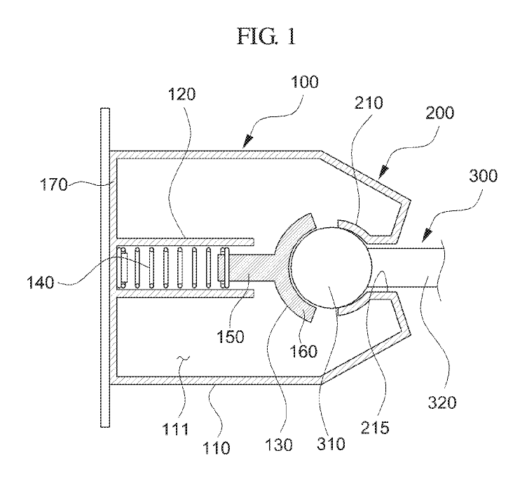

The gist of the present invention relates to an elastic joint structure that is capable of 6 degrees of freedom movement. In FIG. 1, individual components are shown in sections in order to illustrate the basic concept of the mechanism of a joint apparatus according to the present invention.

A joint apparatus according to a first embodiment of the present invention includes a joint means 100, an elastic means 200, and a joint connection means 300, as shown in FIG. 1.

The joint means 100 includes a joint body 110, an reception member 130 located inside the joint body to accommodate the joint connection means 300, and a guide member 120 configured to guide and support the reception member 130 inside the joint body 110. The guide member 120 is fastened to a base plate 170, may be configured in a tubular form on that apart of the reception member 130 can be inserted thereinto, and contains an elastic element 140 so that the inserted reception member 130 can perform rectilinear reciprocal movement. The reception member 130 may include a receptor 160 configured to accommodate the joint connection means 300 while coming into contact with the joint connection means 300, and a receptor support 150 configured to be inserted into the guide member 120.

The joint connection means 300 includes a joint connection means head 310 and a joint connection rod 320, and is held by the elastic means 200 in radial directions, including upward, downward, leftward and rightward directions. The elastic means 200 may be integrated with one end of the joint body 110, or may be coupled to one end of the joint body 110 through screw fastening or welding. The other end of the joint body 110 may be coupled to the firm base plate 170 by screw fastening or welding, or the other end of the joint body 110 may be integrated with the firm base plate 170. The guide member 120 having a smaller diameter, which is perpendicularly fastened to the base plate 170, is formed in the internal space of the joint body 110, and the elastic element 140 is inserted into the guide member 120. One end of the elastic element 140 is fastened to the base plate 170, and the other end of the elastic element 140 is fastened to one end of the receptor support 150.

The joint connection means 300 including the joint connection rod 320 and the joint connection means head 310 is inserted into an elastic means opening 215 from the outside of the joint body 110 to the inside of the joint body 110, i.e., from the right side of FIG. 1 to the left side of FIG. 1. In this case, the elastic means 200 receives the joint connection means head 310 while the space of the elastic means opening 215 is being widened by insertion force, and then presses and holds the rear half portion of the joint connection means head 310. The front half portion of the joint connection means head 310 is held by the receptor 160 placed at a proximal location. In this case, the receptor 160 and the receptor support 150 are pushed in a direction in which the elastic element 140 is compressed, in which case the anti-elastic force of the compressed elastic element 140 balances the anti-elastic force of the elastic means 200, and thus the pushed and inserted receptor 160 and the joint connection means head 310 are held and maintained in a predetermined space. As shown in FIG. 1, the joint connection means head 310 may be formed in a spherical shape, in which case the receptor 160 may be formed in a concave semi-spherical shape. It will be apparent that the joint connection means head 310 and the receptor 160 may be formed in any shape in which they can be coupled to each other in a male and female coupling manner or in an engaged manner.

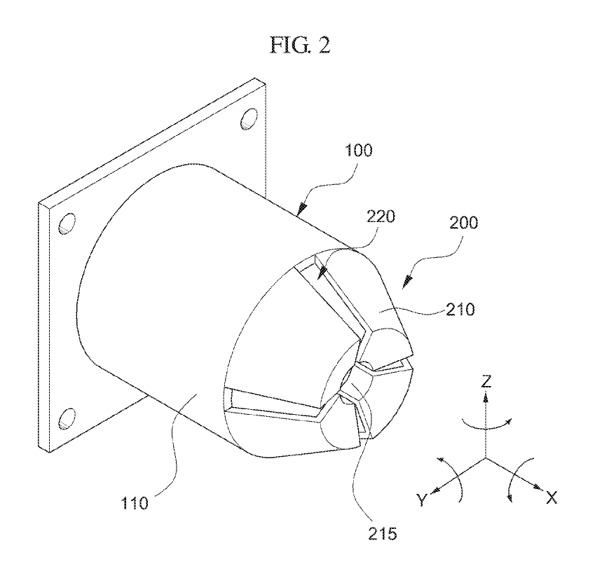

The guide member 120 maintains the receptor 160, the receptor support 150, and the elastic element 140 in a line as much as possible. Furthermore, a spatial gap that is generated in accordance with the difference between the width of the guide member 120 and the width of the receptor support 150 determines the range of accommodation of the slopes of the receptor 160 and the joint connection means head 310. This structure is characterized in that in particular, cutout slits having a width equal to that of the receptor support 150 are formed to a predetermined length in the side of the guide member 120 and thus the range of accommodation of the slopes can be further extended. In contrast, the range of the axial linear movement of the receptor 160 is determined by the difference between the length of the guide member 120 perpendicularly extending from the base plate 170 and the total length of the elastic element 140 and the receptor support 150 extending from the base plate 170. In particular, the contact surfaces of the elastic means 200 and the joint connection means head 310 or joint connection rod 320 and the contact surfaces of the joint connection means head 310 and the receptor 160 enable sliding movement, and thus the joint connection means head 310 and the joint connection rod 320 enable free axial rotation (rotational movement around the X axis in FIG. 2). From the standpoint of the joint connection means head 310, the receptor 160 and the components connected adjacent thereto, the above structure enables three spatial linear movements (rectilinear translational movements in the X-axis, Y-axis and Z-axis directions in FIG. 2) and three rotational movements (rotational movements around the X axis, the Y axis, and the Z axis in FIG. 2) when force is applied, enables return to original locations when applied force is removed, or enables responsive movements to new locations when applied force is changed.

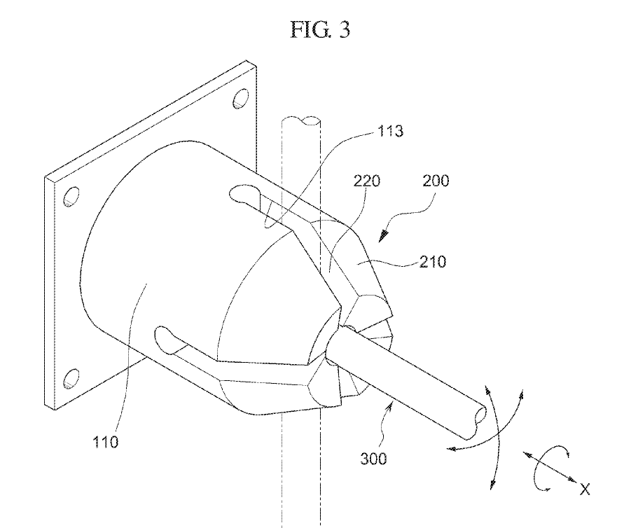

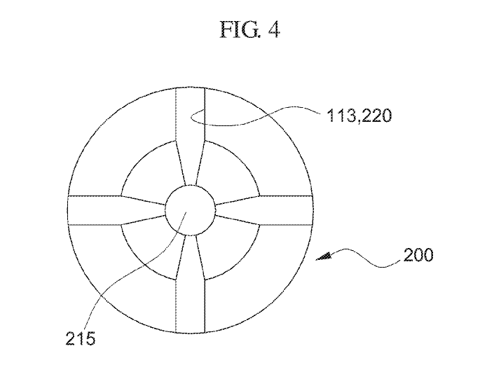

As shown in FIG. 2, the elastic means 200 may be separated by a plurality of elastic means cutout slits 220, and may be composed of a plurality of elastic means units 210. As shown in FIG. 3, a plurality of joint body cutout slits 113 is formed through the joint body 110. FIG. 4 is a front view of the joint apparatus showing the elastic means cutout slits 220 and the joint body cutout slits 113 of FIGS. 2 and 3.

When the elastic means 200 is composed of the plurality of elastic means units 210, as shown in FIGS. 2 and 3, the mounting of a joint is described. The joint connection means head 310 and the joint connection rod 320 are pushed and inserted into the elastic means opening 215, and are mounted into the joint body 110 while the gaps on the entrance side of the elastic means units 210 are being extended. In this case, the elastic means units 210 are pushed toward the receptor 160, and thus the elastic means opening 215 is widened. Once the joint connection means head 310 has been inserted into the joint body 110, the elastic means units 210 are returned to their original locations. In contrast, when the joint connection means head 310 is pulled in the opposite direction, the elastic means units 210 are bent in the opposite directions, and thus the gaps on the entrance side are extended in the opposite directions and the opening 215 is also widened in the opposite directions. Alternatively, when the joint connection means head 310 is bent in a perpendicular direction, first side ones of the entrance portions of the elastic means units 210 are bent in a direction in which they are pushed and inserted and second side ones of the entrance portions of the elastic means units 210 are bent in a direction in which they are extended to the outside, and thus the elastic means opening 215 may be widened and the joint connection means head 310 may be separated from the elastic means 200 and the receptor 160.

The elastic means 200 holds the rear side of the joint connection means head 310 and a part of the joint connection rod 320 near the joint connection means head 310 over an overall radial range of 360 degrees around the X axis. Slight gaps are present among the individual elastic means units 210. When the joint connection means head 310 or receptor 160 is greatly moved in a specific direction, the gaps among the plurality of elastic means units 210 are extended. When these gaps are extended, the joint connection means head 310 and the receptor 160 can be separated through the extended gaps between the elastic means units 210. In the state in which the joint connection means 300 has been inserted into the joint body 110, the joint connection means 300 can perform three-axis rectilinear translational movements and three-axis rotational movements in response to external force in the state of being supported by the elastic member 200. Referring to FIGS. 2 and 3, the joint connection means 300 is shown as being able to perform rectilinear translational movements in the X-axis, Y-axis and Z-axis directions and rotational movements in rotation directions around the X axis, the Y axis and the Z axis with respect to the joint body 110.

As shown in FIG. 1, a space portion 111 is formed inside the joint body 110, the base plate 170 is formed at one end of the joint body 110, and the elastic means 200 is provided on the other end of the joint body 110. The elastic means 200 may be formed to be integrated with the joint body 110, or may be formed to be fastened to the other end of the joint body 110 by a fastening means (not shown).

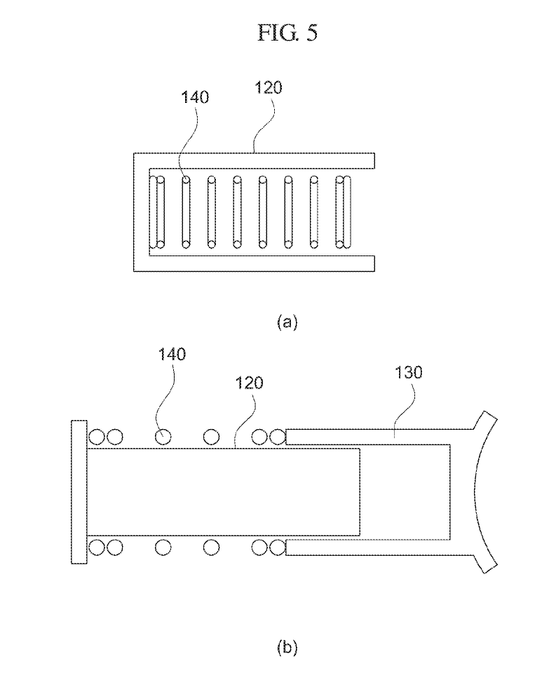

Meanwhile, the guide member 120 formed inside the joint means 100 protrudes perpendicularly from the base plate 170 toward the elastic means 200 inside the joint body 110, as shown in FIG. 1. The guide member 120 may be formed in a tube shape so that it can accommodate and guide the protruding receptor support 150 of the reception member 130, as shown in FIG. 5(a). In this case, the elastic element 140 is provided inside the tube-shaped guide member. Furthermore, the guide member 120 may be formed in a column shape so that it can be inserted into the depressed receptor support 130 and guide the receptor support 130, as shown in FIG. 5(b). In this case, the elastic element 140 is provided outside the column-shaped guide member.

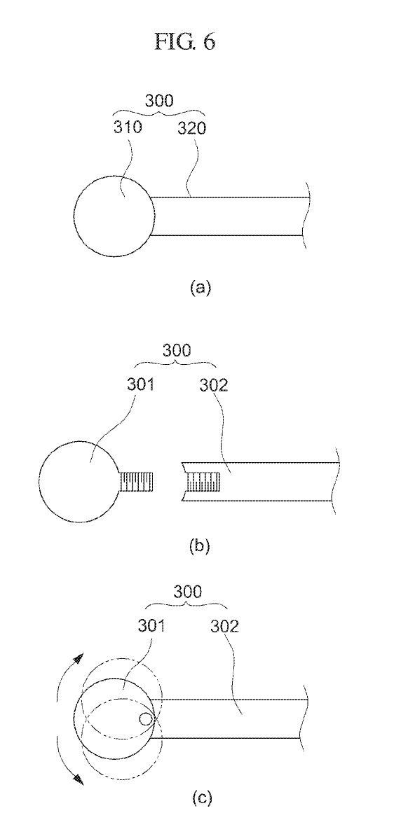

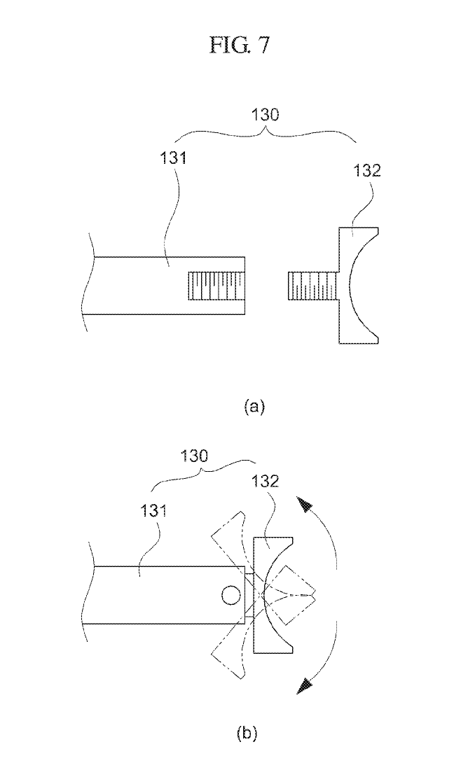

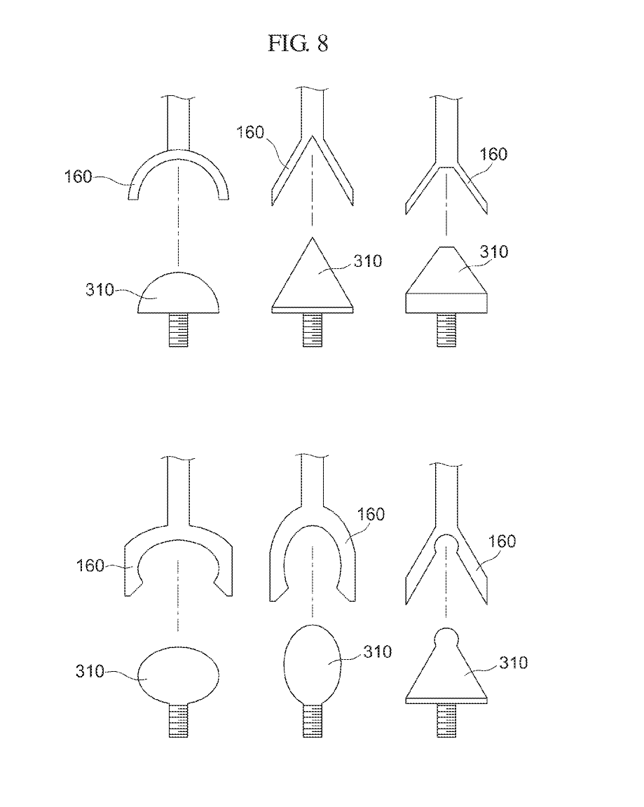

FIGS. 6 and 7 show an embodiment of a joint connection means 300 having a spherical head and an embodiment of an reception member 130 accommodating the joint connection means 300, respectively. A male spherical head 310 or a female receptor 160 may be configured in an integrated form, as shown in FIGS. 1 and 6a. Alternatively, the male spherical head or female receptor may be formed in such a way that two members 301 and 302 (or 131 and 132) are screwed into each other at an end or center of a connection rod as desired, as shown in FIGS. 6b and 7a. Meanwhile, the spherical head or receptor head 310 may be configured to have various shapes including not only a sphere, a cone, a truncated cone, and an ellipse but also polygons, such as a disk, a triangle, a rectangle and the like, as shown in FIG. 8. The spherical head or receptor head 310 may be configured in a form in which a specific male head can be engaged with a female head paired with the male head and contact surfaces thereof can be engaged with each other. The contact portion of the joint connection means 300 and the contact portion of the reception member 130 may be formed in corresponding shapes, and thus may be formed such that they can make smooth sliding contact with each other through male and female coupling. Meanwhile, the male head 301 and the connection rod 302 or the female head 132 and the connection rod 131 may be coupled to each other through hinge coupling, as shown in FIGS. 6c and 7b.

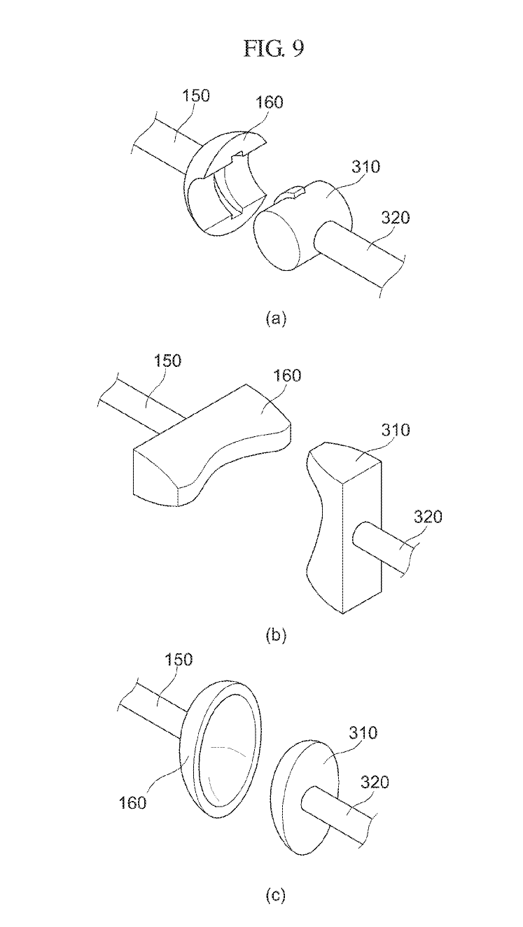

The female heads 301 and 132 may be engaged with each other in a pairwise fashion. A coupling surface may form various coupling surfaces, such as a plane, a male-female spherical surface, a male-female depressed and projected surface, a male-female triangle, a male-female diamond, etc. In FIG. 9, hinge heads (see FIG. 9a), saddle heads (FIG. 9b), and ellipsoidal heads (see FIG. 9c) in a coupling region of a human body joint are shown as examples of the male and female heads 301 and 132. In this case, the male and female heads may be the receptor 160 and the joint connection means head 310, respectively, or may be the joint connection means head 310 and the receptor 160, respectively.

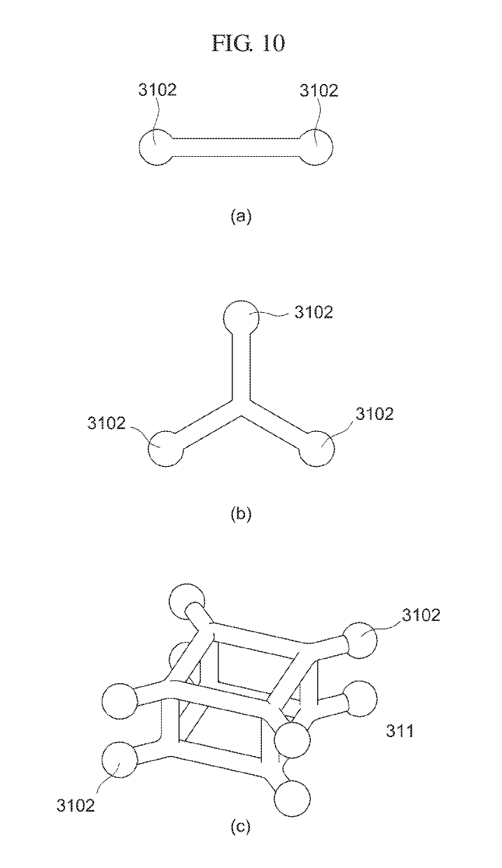

The connection rod 320 is a rod that connects the joint means 100 and the joint means 100. The connection rod 320 may be basically formed in a cylindrical shape having a circular cross section, or may be formed in various shapes having other specifically shaped cross sections. The female receptor head 132 and the male spherical head 301 may be formed at both ends of the connection rod 320 in various combinations. FIG. 10a shows an example in which paired male spheres 3102 are formed at both ends of a connection rod, respectively. Female receptors may be formed at both ends of a connection rod, or a male sphere and a female receptor may be formed on the one and other ends of a connection rod, respectively. The connection rod may form a one-dimensional (1D) linear shape (see FIG. 10a), may form a 2D arrangement (see FIG. 10b) in which the heads of the rod can form angles with each other, or may form a 3D structure (see FIG. 10c), a multi-axis interfacing rod structure. Meanwhile, the connection rod may have a circular cross section, as shown in the examples of FIG. 10, or may have a polygonal cross section, such as an elliptical cross section, a triangular cross section, a rectangular cross section, or the like, as desired. This connection rod may be fitted into one or more other connection rods in a screw manner, and thus may form structures having various shapes. Furthermore, the connection rods and the connection rod structures may perform various 6 degrees of freedom joint functions through combination with the elastic means units.

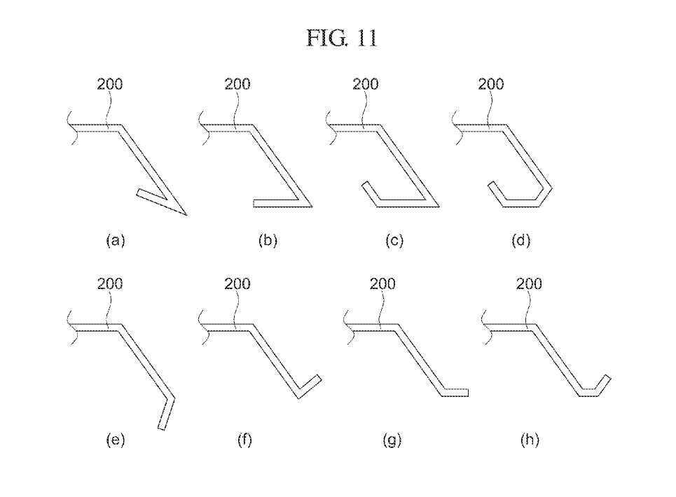

FIG. 11 shows embodiments showing various shapes of the elastic means 200 (the elastic means unit 210 or elastic means unit assembly), which is a key component of a joint structure. That is, there are shown various examples in which only the upper portions of the longitudinal sections of the elastic means 200 of the joint apparatus 1 based on a symmetrical axis along a longitudinal direction are illustrated. In each longitudinal section of FIG. 11, the upper portion of the elastic means 200 is fastened to the joint body 110 of FIG. 1 and not moved by external force, and a center portion extending downward from the upper portion of the elastic means 200 at a predetermined angle is configured to be bent by external force, particularly in the lateral direction of the diagram. The lower portion of the elastic means 200 extending from the center portion at a predetermined angle while being bent may be additionally bent by external force in a lateral direction. The elastic means units 210 may be formed in the combination of various angles among an upper portion, a center portion and a lower portion, as shown in FIG. 11. The elastic means units 210 may be formed in a structure in which they have a plurality of center portions and are bent at various angles, like in a structure including an upper portion, a first center portion, a second center portion, and a lower portion, as shown in FIG. 11h.

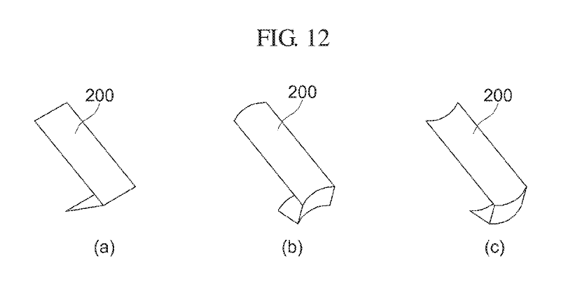

FIG. 12 shows only the center portion and lower portion of each elastic means unit 210. FIG. 12a shows a case in which the surfaces of an elastic means 200 are planar, FIG. 12b shows a case where a curved surface is sequentially changed to a convex surface, a concave surface and a convex surface in a direction from a center portion to a lower portion, and FIG. 12c shows a case in which a curve surface is sequentially changed from a concave surface, a convex surface and a convex surface in a direction from a center portion to a lower portion. As described above, the surfaces of each elastic means unit 210 may be configured in the arrangement of one or more planar surfaces, one or more concave surfaces, and one or more convex surfaces according to the number of bent portions, as desired.

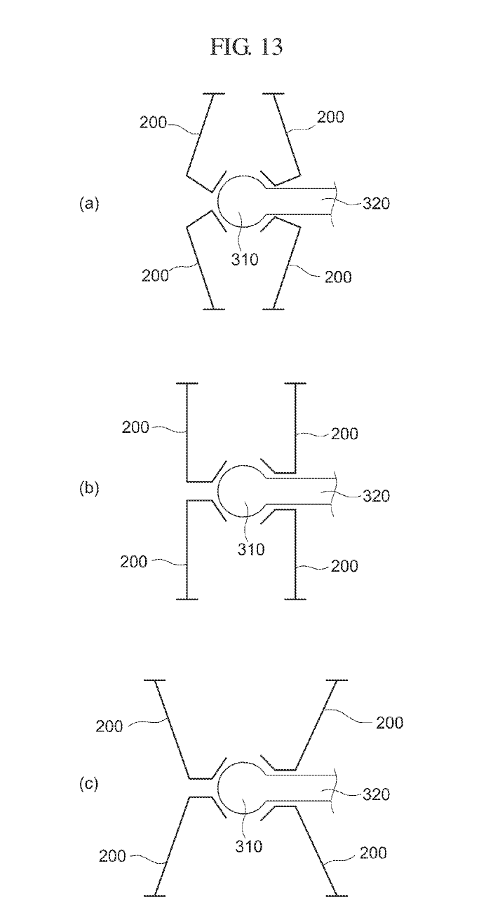

Various embodiments in which spherical joint connection means heads 310 are mounted into elastic means 200 having various shapes are shown in FIG. 13. These embodiments are examples to which an elastic means 200 that is bent to form an upper portion, a center portion and a lower portion is applied. One end of the elastic means 200 is coupled to the joint body 100, and the other end thereof protrudes to the central axis of the space portion 111 and holds the joint connection means 300.

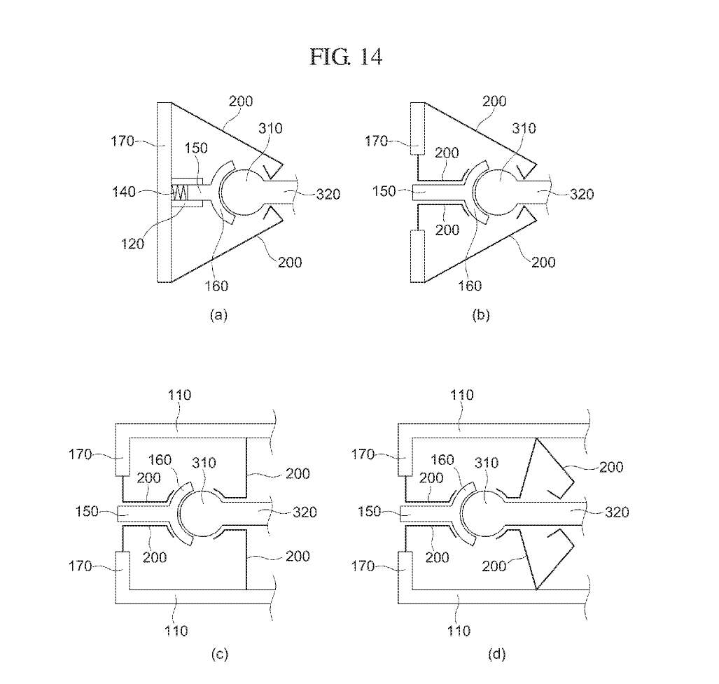

Various embodiments in which the above-described components are combined into a joint structure are shown in FIG. 14. FIG. 14a shows a configuration in which an elastic means 200 mounted on a closed-type mounting base plate 170 and having three bent angles directly holds the spherical joint connection means head 310 and joint connection rod 320 of a joint connection means 300 without requiring the joint body 110 of FIG. 1 and the movement of a receptor head 160 and a receptor support (a receptor rod) 150 is controlled by an elastic element 140 and a guide member 120 mounted on the mounting base plate 170. Meanwhile, FIG. 14b shows a configuration in which an elastic means 200 having one or more bent angles is mounted in the center portion of a mounting base plate 170 having a hole in the center portion thereof without requiring the joint body 110, the elastic means holds a receptor head 160 and a receptor rod 150, and the elastic means 200 radially and directly coupled to the outside portion of the mounting base plate 170 and having three bent angles holds a spherical joint connection means head 310 and a joint connection rod 320. FIG. 14c shows a configuration in which a mounting base plate 170 and a joint body 110 are integrated with each other, unlike in the configuration of FIG. 14b, and an elastic means 200 mounted on the inner circumferential surface of the joint body 110 holds a spherical joint connection means head 310 and a joint connection rod 320. FIG. 14d shows a structure in which two or more elastic means configured to hold a spherical joint connection means head 310 and a joint connection rod 320, like in configuration of FIG. 14c, and thus can more stably hold the spherical joint connection means head 310 and the joint connection rod 320.

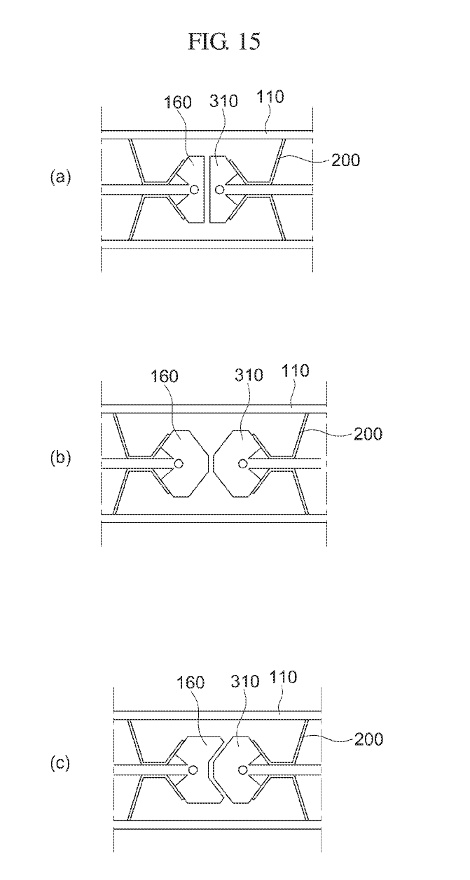

FIG. 15 shows embodiments in which a joint connection means head 310 and a receptor 160 have various shapes, not spherical shapes. FIG. 15a shows an example in which a wide planar contact surface is present between two heads, FIG. 15b shows an example in which a significantly narrow contact surface is present, and FIG. 15c shows an example in which a contact surface has a depressed and projected shape.

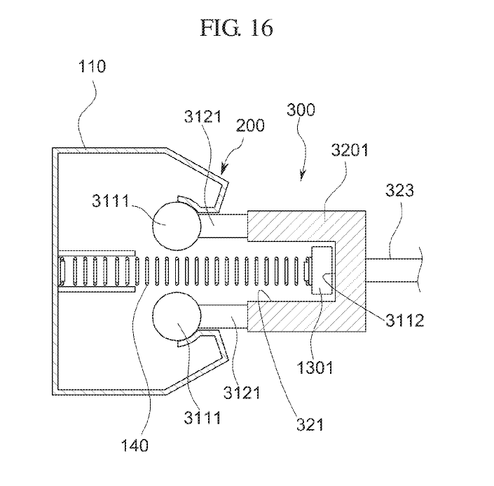

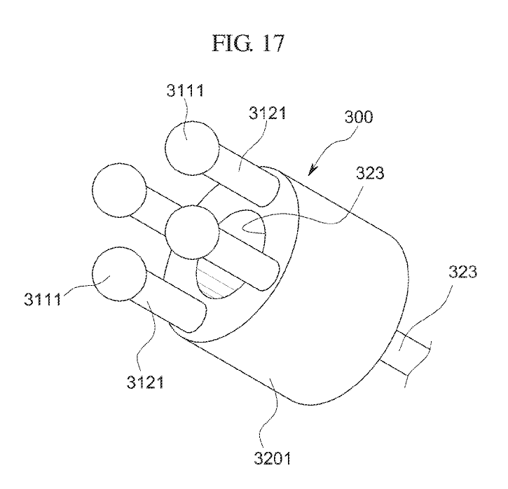

FIGS. 16 and 17 show an embodiment of a joint apparatus 1 in which two or more joint connection means heads 3111 are formed at one end of a joint connection means 300. This joint apparatus 1 is basically the same as the joint apparatus 1 of FIG. 1 in terms of concept, but is different only in that the structure of the joint connection means 300 functions as the guiding cylinder of the elastic element 140 and thus the relatively long guide member 120 is not necessarily required, unlike in FIG. 1. In this embodiment, the joint connection means 300 has a column-shaped joint connection means body 3201, the plurality of joint connection means heads 3111 are connected to one end of the joint connection means body 3201 by joint connection means links 3121, an auxiliary connection rod 323 is connected to the other end of the joint connection means body 3201, and the auxiliary connection rod 323 is responsible for the function of the joint connection rod 320. Furthermore, an insertion hole 321 configured to accommodate the contact element 1301 of a joint means is formed in the one end side portion of the joint connection means body 3201 to which the joint connection means heads 3111 are connected. In the state in which the contact element 1301 of the joint means has been inserted into the insertion hole 321 and has come into contact with an insertion hole bottom 3112, the plurality of joint connection means heads 3111 are inserted into and accommodated in a joint body 110 in the state of being supported by the elastic force of an elastic means 200. When external force, such as pulling force or bending force, is applied to a joint apparatus, particularly the joint connection means 300, any one of the plurality of joint connection means heads 3111 may be separated from the joint body 110, and two or more joint connection means heads 1111 may be separated. In this case, when one joint connection means head 3111 starts to be separated from the joint body 110, the remaining other joint connection means heads 3111 do not receive restoring force from the elastic member 200 on a partial side thereof, and thus are easily and sequentially separated.

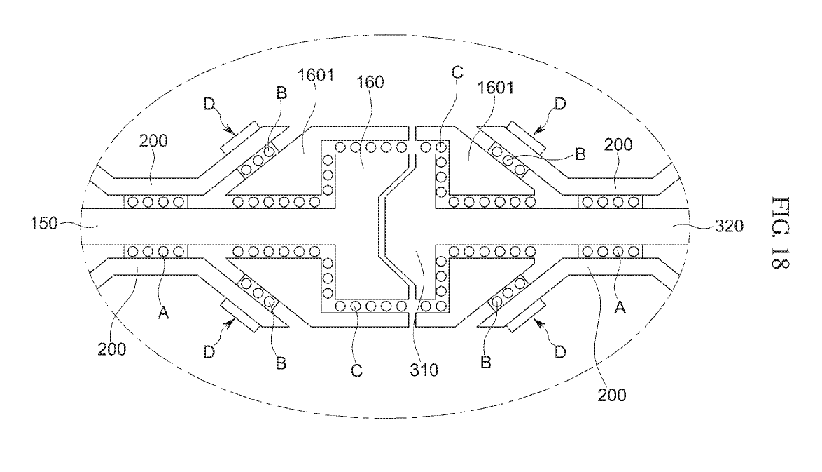

FIG. 18 shows a joint apparatus 1, in which 6 degrees of freedom movements are generated by a head contact surface and a connection rod, as an application of the embodiment of FIG. 15c in which the contact surface between the two heads has a depressed and projected shape. Portions A, B and C of FIG. 18 show arrangements of ball bearings. The ball bearing arrangement A is located between a rod-shaped receptor support 150 and an elastic means 200 or between a joint connection rod 320 and an elastic means 200, and allows male and female heads (i.e., a receptor 160 and a joint connection means head 310) and the joint connection rod 320 to rotate on their central axis. The ball bearing arrangement B is located between the outside body 1601 of the male and female heads 160 and 310 and the elastic means 200, and the ball bearing arrangement C is located between the outside body 1601 of the male and female heads 160 and 310 and the male and female heads. Even when only one of the bearing arrangements A, B and C or only any one of pairs of A and B, A and C, and B and C is present, the rotating force of a shaft can be transferred from a head on one side to a head on the other side. In this structure, the male and female heads are coupled to each other and thus a power shaft is rotated on its axis, and the male and female heads and connection rods enable the force of a rotating shaft to be transferred to a head on the other side while allowing movement to some extent in all the directions of 6 degrees of freedom.

Meanwhile, a plurality of blade-shaped surfaces D protruding from the surface of the outside body of the male and female heads may be located in gaps between a plurality of elastic units radially located, and thus may prevent the outside body of the male and female heads from being rotated. In this case, the head outside body is not rotated, and only the rotating shaft and the ball bearing arrangement C are rotated.

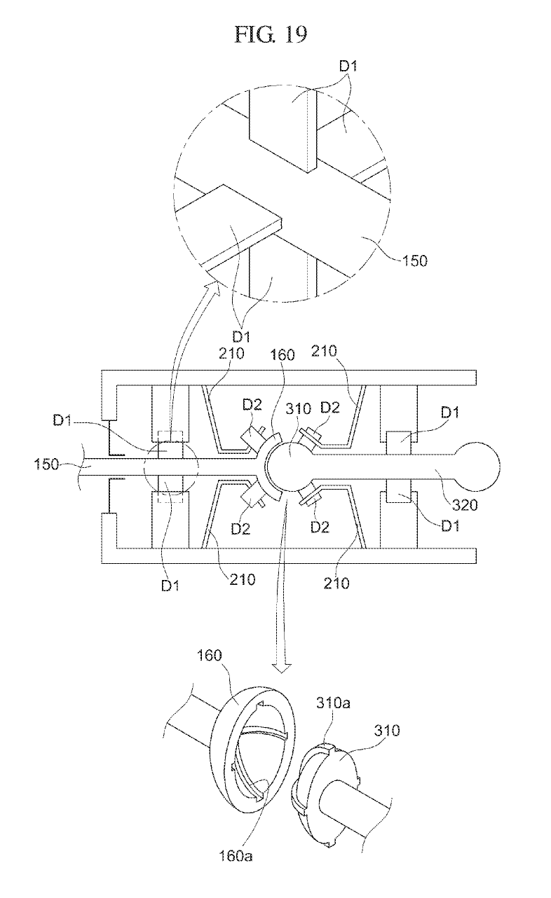

FIG. 19 shows the configuration of the rotation prevention blades D of FIG. 18 in greater detail. As shown in FIG. 19, a spherical joint connection means head 310 and a receptor head 160 are coupled to each other in the state in which protrusions 310a and depressions 160a have been formed on the contact surfaces of the spherical joint connection means head 310 and the receptor head 160. Accordingly, when a connection rod 320 or 150 on one side generates rotating force, the rotating force may be transferred to a connection rod 150 or 320 on the other side. However, one or more blades D1 protrude perpendicularly from the outer circumferential surfaces of the connection rods and are located in gaps between elastic units 210 protruding from a joint body 110 toward the center, thereby stopping the rotation of the rotating shaft on its axis. Furthermore, one or more blades D2 may protrude perpendicularly from the outer circumferential surfaces of the spherical joint connection means head 310 and the receptor head 160 and may be located in gaps between a plurality of adjacent elastic means units 210, thereby stopping the rotation of the connection rods 150 and 320 and the male and female heads 160 and 310 on their central axis.

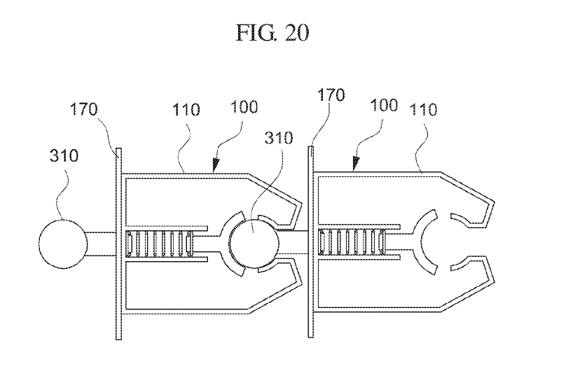

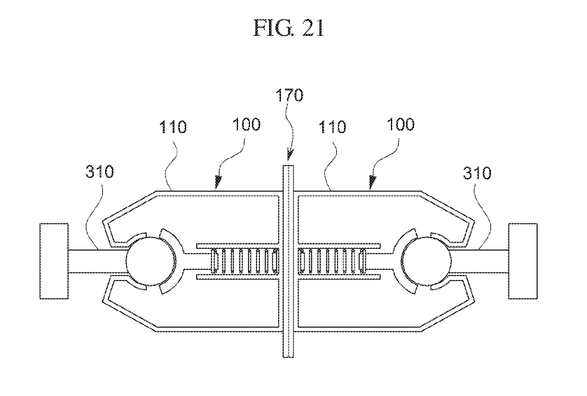

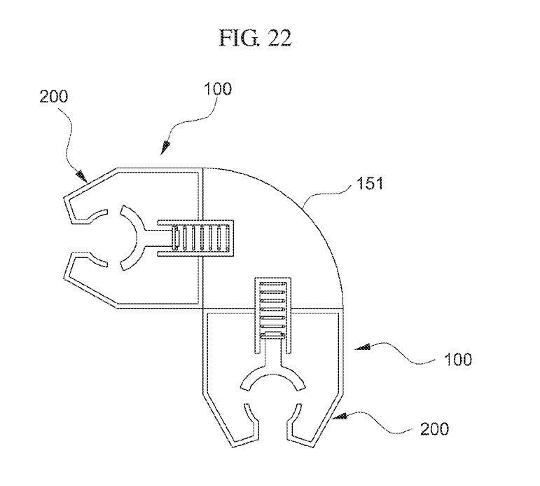

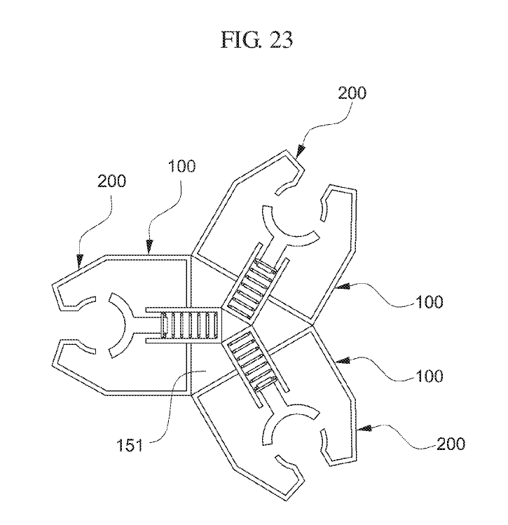

FIG. 20 shows a state in which joint apparatus units in each of which a spherical joint connection means head 310 is added to the joint apparatus of FIG. 1 on the other side (the left side of the drawing) of the base plate 170 are connected in series. Meanwhile, FIG. 21 shows a structure in which the joint apparatuses of FIG. 1 are symmetrically provided in a pairwise fashion while sharing a base plate 170 and thus a plurality of joint apparatus units may be connected in series via connection rods at both ends. FIG. 22 shows an embodiment in which two joint apparatus units are disposed in the state of being bent at right angles while sharing a connection link 151 having a quarter fan-shaped section and, thus, can operate as a single joint structure. FIG. 23 shows an embodiment in which three joint apparatus units are disposed at angular intervals of 120 degrees while sharing a connection link 151 having a triangular cross section and, thus, can operate as a three-directional joint structure. As described above, the joint apparatus units may form a 1D joint apparatus structure, a composite 2D joint apparatus structure or a composite 3D joint apparatus structure according to the shape of the connection link 151.

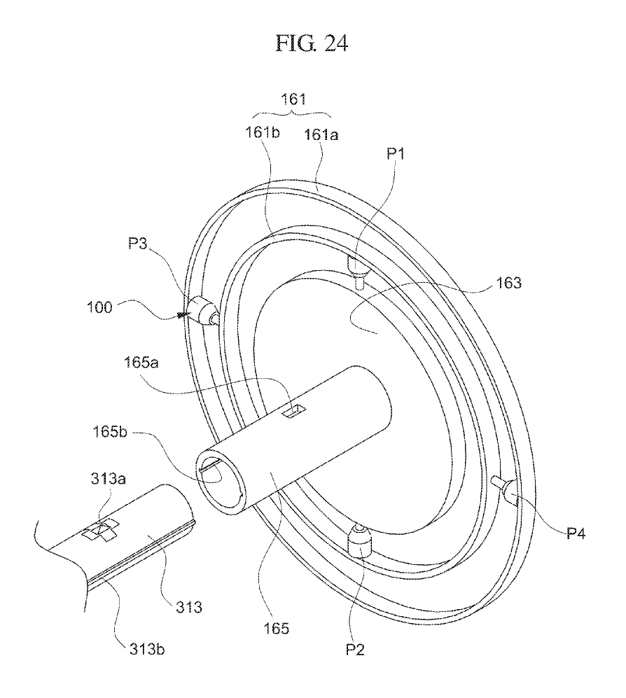

FIG. 24 shows an example in which a plurality of joint apparatuses is applied particularly to a joint region of a dummy or a doll having a human body shape. When a dummy joint is subjected to a pushing action, a pulling action, a bending action, a hitting action, or a twisting action performed by a user, the dummy joint needs to show a reaction similar to that of a corresponding region of a human body. That is, it is necessary to receive the realistic feedback of the reaction of the dummy to external force. The shoulder joint portion of a human dummy is described as an example. A right ring-shaped joint structure corresponding to a torso-side joint and an arm-side ring-shaped joint structure (on the left side of FIG. 24) coupled to the right ring-shaped joint structure are shown in FIG. 24. Each of the ring-shaped joint structures includes two concentric holder rings (161; 161a and 161b), a disk 163 located inside the inner holder ring 161b, a connection tube 165 configured to protrude vertically from the center of the disk, two joint means (100; i.e., P3 and P4) configured to connect the outer holder ring 161a and the inner holder ring 161b, and two joint means (100; i.e., P1 and P2) configured to connect the inner holder ring 161b and the disk 163. The arm-side joint has the same structure as the torso-side joint.

Components provided to connect the two ring-shaped joint structures include the connection tube 165 of the right ring-shaped joint structure, and the connection rod 313 of the left ring-shaped joint structure configured to be inserted into and coupled to the connection tube 165. A fitting hole 165a is formed in the connection tube 165, and guide grooves 165b are formed through the inner surface of the connection tube. Furthermore, an elastic protrusion 313a configured to be fitted into and coupled to the fitting hole 165a of the connection tube 165 and a guide protrusion 313b configured to be guided through the guide groove 165b are formed on the surface of the connection rod 313.

In an application of a human body joint, holder rings 161, a disk 163, and joint means 100 (P1, P2, P3, and P4) may be symmetrically mounted on one side of the connection rod 313, as shown in FIG. 24. In the state in which the connection rod 313 protruding from this structure has been combined with the connection tube 165 into a single connection rod, when this connection rod is held and bent in a vertical direction, external force is applied to the joint means P1 and P2, and thus the disk 163 may be separated from the inner holder ring 161b. Furthermore, when a hit is applied to the connection rod in a vertical direction, external force is applied to P3 and P4, and thus the inner holder ring 161b may be separated from the outer holder ring 161a. In a similar manner, when P3 and P4 are operated by bending force in a lateral direction, the inner holder ring 161b may be separated from the outer holder ring 161a. When hitting force is applied in a lateral direction, P1 and P2 are operated, and thus the disk 163 may be separated from the inner holder ring 161b.

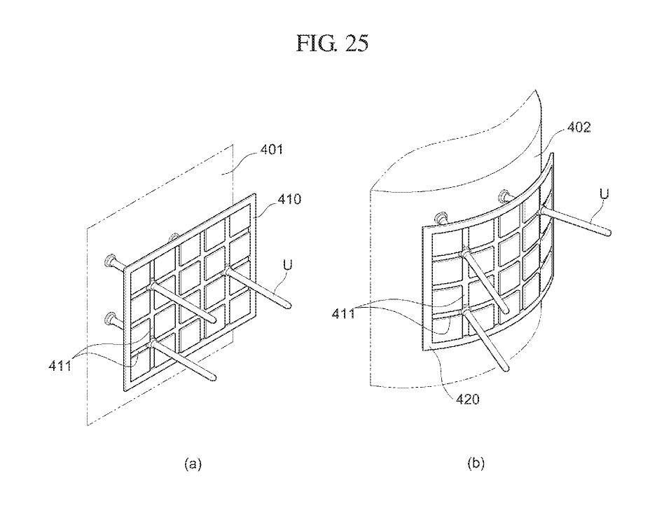

FIG. 25 shows an example of martial arts training equipment, which is mounted and used on a wall or a curved column, as an application of joint units. Training units which have the form of a cylinder made of wood, metal, plastic, or flexible material, such as rubber, surrounding one of wood, metal, and plastic, and whose first ends are spherical heads and whose second ends have spherical or other shapes are basically used. The training units U include flanges having elastic joint units at center portions thereof and fastening screw holes on the peripheral portions thereof. In FIG. 25a, the first ends of the training units U are fastened to a wall 401, a high-rigidity planar plate 410 is attached to the second ends of the training units U, grid-shaped grooves 411 are formed in the planar plate 410, the flanges of the training units U are inserted into the grooves 411, and the training units U are slid to required locations and fastened by screws. A plurality of training units may be mounted on a planar plate, and this structure may be used as training equipment. FIG. 25b shows an example of curved training equipment to which a curved plate 420 mounted on a curved column 402 instead of a wall is attached.

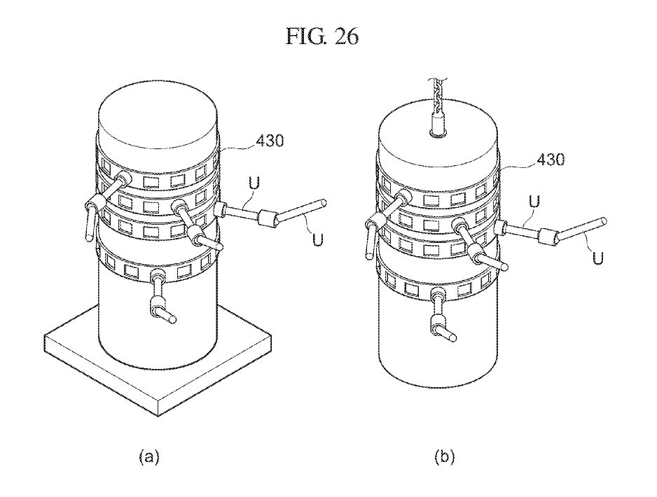

FIG. 26 shows Muk Yan Jong (see FIG. 26a) or punching bag-shaped training equipment (see FIG. 26b) that is formed by mounting one or more belts 430 on a column-shaped object and then mounting the training units U on the belts 430 at desired locations. These cases are characterized in that a column-shaped object or a punching bag body, and a column supporting the column-shaped object or a chain hanging the punching bag body can be replaced with the joint apparatus according to the present invention and then can be used.

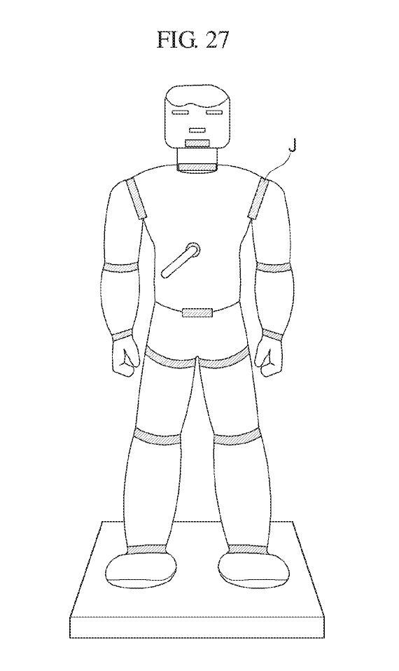

Meanwhile, FIG. 27 shows an example of a human body doll that is constructed by connecting respective parts of a model of a human body by the joint apparatuses 1 according to the present invention. In this example, the respective parts are connected by the joint apparatuses 1 in a plurality of hatched joint regions J. The training of martial arts can be effectively performed using this model of a human body. In this case, it will be apparent that the joint apparatus according to the present invention may he applied not only to the portions illustrated in FIG. 27 but also to all types of human body joints and then may implement joint functions.

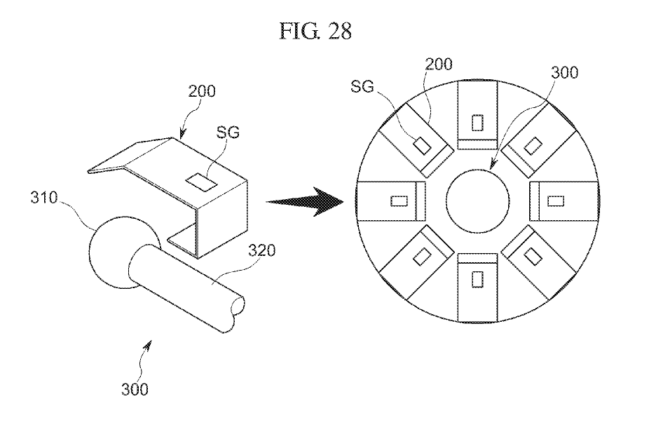

Furthermore, a load cell sensor may be constructed by mounting a strain gauge SG on a part of the joint apparatus 1 of the present invention, for example, a surface of the elastic means 200, as shown in FIG. 28, and then connecting the strain gauge SG to a Wheatstone bridge circuit, and then may determine external force or strain applied to the joint apparatus in real time through the measurement of the external force or strain.

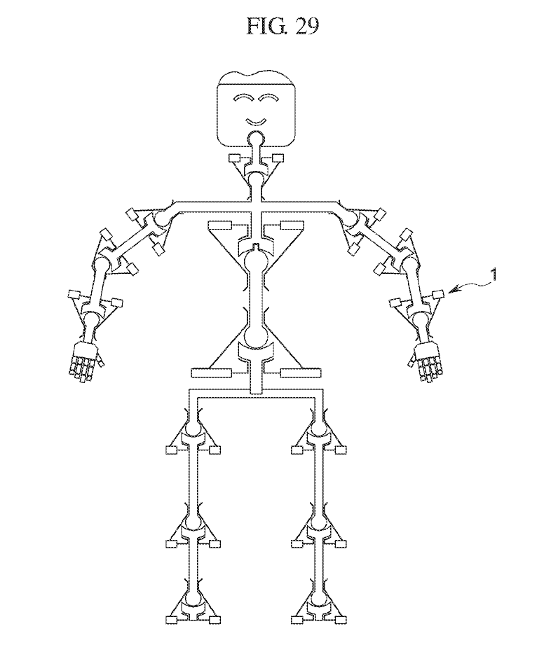

FIG. 29 shows an example in which the joint apparatuses according to the present invention are applied to a construction toy that simulates a human body. It can be seen that respective parts of the toy are constructed by assembling and connecting the joint apparatuses 1 of the present invention by connecting rods.

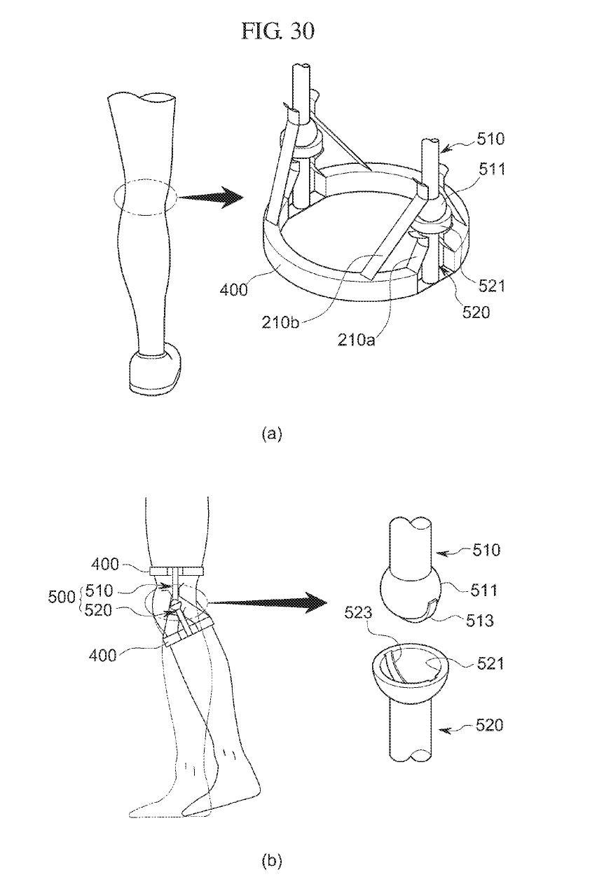

FIG. 30 shows an embodiment of a muscle strengthening exoskeleton structure using the joint apparatuses according to the present invention. This muscle strengthening exoskeleton structure may be constructed by perpendicularly connecting two joint units, and then may be used to support a corresponding joint region of a user who lost and cannot use all or part of the functions of a joint, such as a knee or neck joint (not shown). Referring to FIG. 30a, each joint connection means including a spherical head 511 and each reception member including a receptor head 521 accommodating the joint connection mean are fastened to and mounted on a ring member 400, surrounding a joint region, by elastic members 210a and 210b. The spherical head 511 of each joint may be held by the second elastic member 210b, and the receptor head 521 may be held by the first elastic member 210a. In particular, as shown in FIG. 30b, a male protrusion 513 and a female groove 523 may be formed on the spherical head 511 and receptor head 521 of the joint, respectively, and thus the joint may be bent only in a direction in which the corresponding joint, for example, a knee joint, is bent.

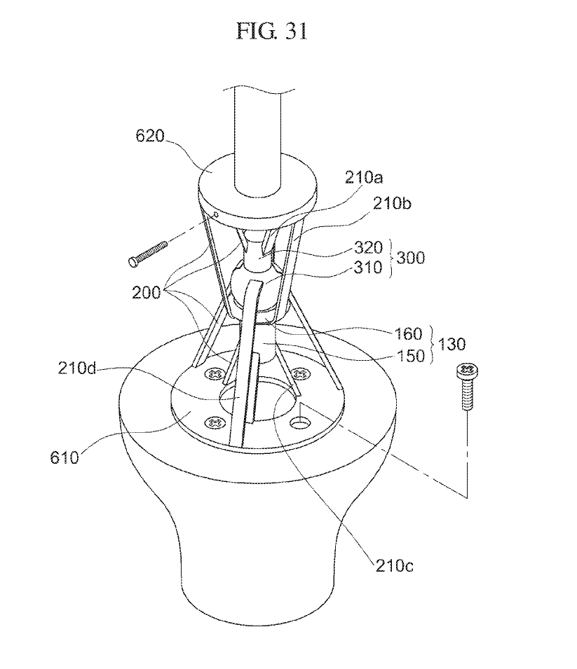

FIG. 31 shows an embodiment of a joint apparatus that connects two bones when a defect occurs in a part of a bone in the internal skeleton structure of a human body. When a joint region between an upper bone shown in the upper portion of the drawing and a lower bone shown in the lower portion thereof does not perform joint functions due to cutting or damage, the lost joint functions should be replaced using an artificial joint structure. A skeleton-mounted ring 620 is mounted on the upper bone through screw fitting, bonding fitting or belt-type fitting. First elastic means 210a are radially disposed along the internal circumferential surface of the skeleton-mounted ring 620, and the first elastic members 210a fasten and hold the joint connection rod 320 of a joint connection means 300. Second elastic means 210b are radially disposed along the outer circumferential surface of the skeleton-mounted ring 620, and the second elastic members 210b hold the rear end of a receptor 160 and a receptor support 150.

In contrast, a skeleton-mounted disk 610 is fastened to and mounted on the upper surface of the lower bone by a fastening and coupling method, such as screw fitting, bonding fitting, or belt-type fitting. Third elastic means 210c are radially disposed along the internal circumferential surface of the skeleton-mounted disk 610, and the third elastic members 210c fastens the lower end of the receptor support 150. Furthermore, fourth elastic means 210d are radially disposed along the outer circumferential surface of the skeleton-mounted disk 610, and the fourth elastic members 210d hold the joint connection means 300, the rear surface of the joint connection means head 310, and a partial surface of the joint connection rod 320.

In this case, the joint connection means head 310 and the receptor 160 are configured to have male and female surfaces, respectively, and maintain sliding contact, and thus a relative angular and linear range between the joint connection rod 320 and the receptor support 150 may be freely changed in an intended direction as desired, like in a joint of a human or an animal. The joint connection rod 320 and the receptor support 150 are simply held by the elastic members 210. Accordingly, when excessive force exceeding the limit of the restoring force of the elastic members 210 is applied, the joint connection rod 320 and the receptor support 150 are separated from each other, or are detached from each other in a serious case, thereby generating an effect identical to that of dislocation. Accordingly, the upper bone and the lower bone perform their original joint functions by means of the skeleton connection artificial joint apparatus using the joint apparatus according to the present invention.

The specific embodiments of the present invention have been described in detail above. However, it will be apparent to those having ordinary knowledge in the art to which the present invention pertains that the spirit and scope of the present invention are not limited to those specific embodiments and various modifications and alterations are possible within orange that does not depart from the gist of the present invention.

Accordingly, the embodiments described above are provided to tell the scope of the present invention to those having ordinary knowledge in the art to which the present invention pertains. Accordingly, it should be appreciated that the embodiments described above are illustrative in all aspects but are not limitative. The present invention is defined by the scope of the claims.

<Description of Reference Numerals of the Drawings>

| | |
|---|---|
| 1: joint apparatus | |
| 100: joint means | 110: joint body |
| 111: space portion | 113: joint body cutout slit |
| 120: guide member | 130: reception member |
| 1301: contact element | 131: first reception member |
| 132: second reception member | 140: elastic element |
| 150: receptor support | 151: connection link |
| 160: receptor | 160a: depression |
| 1601: outside body | 161: holder ring |
| 161a: outer holder ring | 161b: inner holder ring |
| 163: disk | 165: connection tube |
| 165a: fitting hole | 165b: guide groove |
| 170: base plate | 200: elastic means |
| 210: elastic means unit | 210a: first elastic member |
| 210b: second elastic member | 210c: third elastic member |
| 210d: fourth elastic member | 215: elastic means opening |
| 220: elastic means cutout slit | 300: joint connection means |
| 301: first joint connection means | 302: second joint connection means |
| 310, 3111: joint connection | 310a: protrusion |
| means head | |
| 3102: male sphere | 320: joint connection rod |
| 3111: joint connection means head | 3112: insertion hole bottom |
| 3121: joint connection means link | 313: connection rod |
| 313a: elastic protrusion | 313b: guide protrusion |
| 3201: joint connection means body | 321: insertion hole |
| 323: auxiliary connection rod | |
| 400: ring member | 401: wall |
| 402: curved column | 410: planar plate |
| 420: curved plate | 430: belt |
| 511: spherical head | 513: male projection |
| 521: receptor head | 523: female depression |
| 610: skeleton-mounted disk | 620: skeleton-mounted ring |

1. A joint apparatus, comprising:
a joint member;
an elastic member formed on one side of the joint member; and
a joint connection member inserted and mounted into the joint member and configured to directly come into contact with and be held by the elastic member.

2. The joint apparatus of claim 1, wherein a reception member configured to accommodate the joint connection member is disposed in the joint member.

3. The joint apparatus of claim 2, wherein a guide member configured to guide a movement of the reception member is fastened to and mounted in the joint member.

4. The joint apparatus of claim 3, wherein the guide member and the reception member are connected by an elastic element.

5. The joint apparatus of claim 4, wherein an elastic member opening is formed in the elastic member, and the joint connection member is inserted into the joint member via the elastic member opening.

6. The joint apparatus of claim 5, wherein the joint connection member inserted into the joint member is supported through equilibrium of anti-elastic forces of the elastic member and the elastic element, wherein the anti-elastic force of the elastic means and the anti-elastic force of the elastic element act in opposite directions.

7. The joint apparatus of claim 6, wherein contact surfaces of the joint connection member and the reception member have corresponding male and female shapes or corresponding engaged shapes.

8. The joint apparatus of claim 6, wherein the elastic member comprises a plurality of elastic member units, and elastic member cutout slits are formed between the adjacent elastic member units.

9. The joint apparatus of claim 6, wherein the elastic member has one or more bent portions so that the joint connection member can be easily inserted into the joint member.

10. The joint apparatus of claim 1, wherein the elastic member enables elastic deformation with three linear degrees of freedom and three rotational degrees of freedom, and the joint connection member enables three linear degrees of freedom movements and three rotational degrees of freedom movements with respect to the joint member, thereby enabling six degrees of freedom movements.

11. The joint apparatus of claim 1, wherein a composite joint structure having a linear, surface, or three-dimensional structure is formed through coupling of connection links or connection rods.

12. A ring-shaped joint structure, comprising:
two holder rings configured to have different diameters;
a disk configured to have a diameter smaller than those of the holder rings; and
a protrusion configured to protrude perpendicularly from the disk;
wherein by the joint apparatus according to claim 1 the two holder rings are coupled to each other and the holder ring having a smaller diameter is coupled to the disk, and the elastic member of the joint apparatus enables elastic deformation with three linear degrees of freedom and three rotational degrees of freedom.

13. The ring-shaped joint structure of claim 12, wherein the joint apparatus enables three linear degrees of freedom movement and three rotational degrees of freedom movement, thereby enabling six degrees of freedom movement.

14. (canceled)

15. The joint apparatus of claim 1, wherein one or more sensors are mounted on the joint member or the elastic member, and detect applied force or strain.

16-17. (canceled)

18. An artificial joint, comprising:
a first target object;
a skeleton-mounted ring configured to surround and fasten the first target object;
a second target object;
a skeleton-mounted disk configured to fasten the second target object;
a joint connection member located between the first target object and the second target object;
a reception member configured to accommodate the joint connection member between the first target object and the second target object;
a first elastic member radially coupled along an internal circumferential surface of the skeleton-mounted ring, and a second elastic member radially coupled along an outer circumferential surface of the skeleton-mounted ring; and
a third elastic member radially coupled along an internal circumferential surface of the skeleton-mounted disk, and a fourth elastic member radially coupled along an outer circumferential surface of the skeleton-mounted disk;
wherein the first elastic member and the fourth elastic member are coupled to the joint connection member, and the second elastic member and the third elastic member are coupled to the reception member.

19. The artificial joint of claim 18, wherein:
the joint connection member comprises a joint connection member head and a joint connection rod; and
the reception member comprises a receptor and a receptor support.

20. (canceled)

21. The artificial joint of claim 18, wherein:
the first elastic member holds the joint connection rod;
the second elastic member holds the receptor;
the third elastic member holds the receptor support; and
the fourth elastic member holds the joint connection member head.
